(19) United States
(12) Patent Application Publication
(10) Pub. No.: US 2015/0118214 A1
(43) Pub. Date: Apr. 30, 2015

(54) Pharmaceutical composition containing human cyclooxygenase and doxorubicin or doxorubicin analogue, preparation method therefor and use thereof in preparing drugs

(71) Applicant: Xiuying LIN, Anshun, Guizhou (CN)

(72) Inventor: Xiuying Lin, Anshun (CN)

(73) Assignee: Xiuying Lin, Anshun, Guizhou (CN)

(21) Appl. No.: 14/397,860

(22) PCT Filed: Dec. 13, 2012
(86) PCT No.: PCT/CN2012/086605

§ 371 Date: Oct. 30, 2014

(30) Foreign Application Priority Data
May 10, 2012 (CN) 201210142501.4

(51) Int. Cl. A61K 38/44; A61K 36/88; A61K 31/704

(52) U.S. Cl. CPC A61K 38/44; C12Y 114/99001; A61K 31/704; A61K 36/88
USPC 424/94.4

(57) ABSTRACT

Provided are a pharmaceutical composition containing human cyclooxygenase and doxorubicin or a doxorubicin analogue using Salvia plectranthoides (also referred to as rare grass) as a crude drug, a preparation method therefor and the use thereof in preparing drugs for treating nephritis, cholecystitis and gallbladder polyps, tumours or antiphlogistic and analgesic drugs.

### TECHNICAL FIELD

The present invention relates to a natural plant extract from a pharmaceutical composition, in particular relates to a method comprising the native human cyclooxygenase (COX-2), native doxorubicin (atm) or a pharmaceutical composition of doxorubicin substances of natural origin, which contains natural human cyclooxygenase, doxorubicin pharmaceutical compositions of natural or natural substances Class doxorubicin preparation methods thereof, and pharmaceutical compositions comprising this application natural human cyclooxygenase natural doxorubicin or doxorubicin natural origin in the preparation of pharmaceutical substances in the treatment of nephritis, in the preparation and treatment of bladder polyp cholecystitis medicament, in the preparation of a medicament for the treatment of tumors application, or anti-inflammatory in the manufacture of a medicament.

### BACKGROUND

Adriamycin (Doxorubicin) Chemical Name: (2R,4S)-4-(3-Amino-2,3,6-trideoxy-α-L-lyxo-hexopyranosyloxy)-2-hydroxyacetyl-1,2,3,4-tetrahydro-2,5,12-trihydroxy-7-methoxynaphthacene-6,11-dione, inhibits RNA Synthesis of DNA. RNA inhibition against the strongest, broad spectrum anti-tumor, has the role of a variety of tumors, is a cycle non-specific drugs for various growth cycles I have killing effect. Primarily for acute leukemia, acute lymphocytic leukemia and leukemia are valid. Malignant lymphoma can be used as the first medication used interchangeably. A variety of other cancers breast, lung cancer, bladder cancer have a certain effect, and more in combination with other anticancer.

The product is a broad-spectrum anticancer drugs, the body can produce a wide range of biochemical effects, with a strong cytotoxicity, The main mechanism of action is that the product is embedded in DNA synthesis inhibition of nucleic. Clinically for the treatment of acute lymphoblastic leukemia, acute myelogenous leukemia. Hodgkin's and non-Hodgkin's lymphoma, breast cancer, lung cancer, ovarian cancer, soft tissue sarcoma, osteosarcoma, rhabdomyosarcoma, Wilms tumor, neuroblastoma, bladder tumors, thyroid tumor, choriocarcinoma, blue prostate cancer, testicular cancer, stomach cancer, liver cancer. Human cyclooxygenase (cyclooxygenase COX), also known as prostaglandin endoperoxide synthase (Rostaglandenhyperoxidesynthase PGHS), which is the conversion of arachidonic acid to prostaglandins and twenty alkenes rate-limiting enzyme, including COX-2 and COX-1 two different structures and different physiological functions cyclooxygenase, Human cyclooxygenase (COX-2) was discovered in 1988; natural human cyclooxygenase be separated from the plant in 1991. Cyclooxygenase is a natural human protein is an inducible enzyme, the enzyme can act to accelerate the production of a chemical signal, when active inflammation and pain. When the COX-2 activity is inhibited, the pain relief, After activation of COX-2 may be the birth of arachidonic acid, resulting in a variety of prostaglandins, the body involved in many physiological and pathological processes Currently more agreeable view is, COX-2 can promote cell proliferation, inhibition of apoptosis, promote angiogenesis, inhibit immune function mechanisms involved in tumor development and progression.

Although research papers on human cyclooxygenase (COX-2) conducted numerous, but the art has not found a rich source of human cyclooxygenase (COX-2) natural medicinal plants, traditional Chinese medicine approach can be adopted achieve natural doxorubicin (atm) and human cyclooxygenase (COX-2) in the pharmaceutical industry.

References:

Xue Petunia, often red, Yang Lirong, Yanan University (Medical Sciences) 2011 09 Volume 03: “COX-2 and Research Progress of non-small cell lung cancer”,

Zou Ying Ying, Yao Nan, Wang Fang, Gao Qian, Song Jing Ling, Kunming Medical College in 2010 Volume 31 01: “CDX2 and COX2 expression in human colorectal tumor characteristics and with metastasis”;

Huifeng, high Xiaoqin, Li Rong Mountain: Shanxi Medical University 2009 40 Volume 12: “P_{———} (38) MAW/COX- 2 signal transduction pathway in rat kidney ischemic preconditioning second window of protection effect”;

Shi Bo, the Liu Jiang, HE Jian Fang, Dai Licheng, Zhou Jian Fang, Zhao Weifeng, sweet and construction, Central Plains Medical Journal 2007 Volume 34 09: “cyclooxygenase-2 in hepatic failure model of liver injury in rats”;

### SUMMARY OF THE INVENTION

In order to remedy the above deficiencies of the prior art, an object of the invention is to provide a containing natural human cyclooxygenase (COX-2), doxorubicin natural (atm) or a pharmaceutical composition of doxorubicin substances of natural origin, which cyclooxygenase-containing natural human species, the method for preparing a pharmaceutical composition of natural classes doxorubicin or doxorubicin natural substance, and that such natural human cyclooxygenase-containing natural doxorubicin or doxorubicin substances of natural origin The pharmaceutical composition of drugs used in the treatment of nephritis, cholecystitis, and in the manufacture of therapeutic drugs in the bladder polyp, in the manufacture of a medicament for the treatment of tumors, or in the manufacture of anti-inflammatory drugs. The present invention provides a rich human cyclooxygenase (COX-2) and the class of natural or doxorubicin doxorubicin medicinal preparation of a pharmaceutical composition of natural plants, the composition can be achieved using traditional Chinese medicine natural doxorubicin Su (atm) and human cyclooxygenase (COX-2) in the pharmaceutical industry. Avoid the many side effects of synthetic drugs, and has a good therapeutic effect. For purposes of the present invention, a technical solution as follows: A kind of pharmaceutical compositions containing natural human cyclooxygenase (COX-2), a natural doxorubicin (atm) or natural substances class doxorubicin, wherein the raw material of the compositions is the Rare grass (or grass Guizhou Treasure). The above technical solution described in the “composition of the feedstock Rare grass” means: Rare grass whole plant may be used (including the base color) dehydrated, dried and pulverized to obtain a powder; fresh grass or the Treasure product or its extract.

The Rare grass extracts means in the form of an extract:

Velvet made of mashed rare grass fresh goods;

Rare grass decoction;

Powder obtained from concentrated and dried Rare grass decoction;

Ethanol extracts obtained from Rare grass decoction by ethanol extract;

Powder obtained from ethanol extractive of rare grass decoction, and then concentrated and dried.

Organic solvent extracts obtained from rare grass whole plant or decoction by organic solvent extraction; Powder obtained from Organic solvent extracts of rare grass whole plant or decoction, and then concentrated and dried.

Extracts obtained from Rare grass whole plant or decoction by supercritical carbon dioxide extraction; Powder obtained from extracts of Rare grass whole plant or decoction by supercritical carbon dioxide extraction, and then concentrated and dried. Rare grass whole plant extracts essential oil.

Treasure materials other than the grass or extract thereof, which contains natural human cyclooxygenase (COX-2), native doxorubicin (atm) or a pharmaceutical composition of doxorubicin substances of natural origin may also contain pharmaceutical field allowed accessories. For example: as an excipient, flavoring agents or antioxidants of a tablet, as antioxidant or stabilizer in a capsule; flavoring agents when used as oral solution or syrup, stabilizers or antioxidants, as an ointment for external use of the time emulsifying or vaseline; injection time as emulsifiers, solvents, or stabilizers, and the like.

Technical solutions described above rare grass (tentative name) of plant classification position as follows:

Guizhou Rare grass, Guizhoutreasures grass;

Salvia, Salvia plectranthoides Griff Department;

Angiosperm, Angiospermae

Dicotyledoneae, Dicotyledoneae

Resolution spend together subclass Sympetalae

Tube flower Tubiflorae

Verbena suborder, Verbenineae

Lamiaceae, Labiatae

Salvia, Salvia linn.

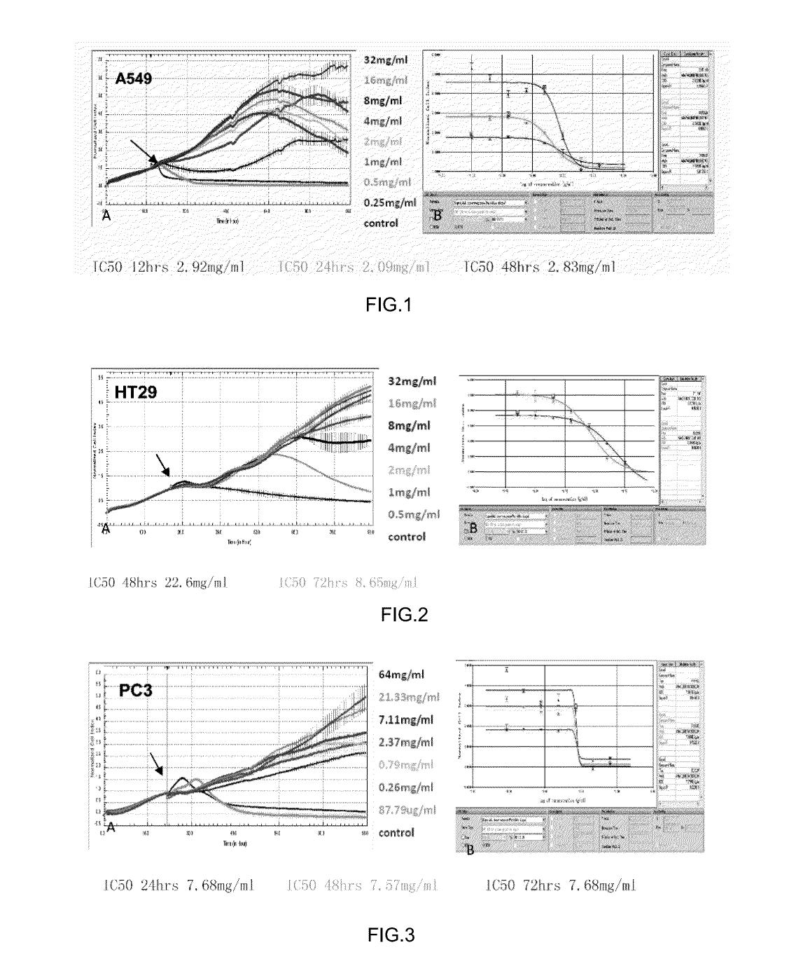

The Treasure of annual or perennial herbaceous grass, fibrous roots, rhizomes erect or prostrate rising Portuguese, Portuguese runners at the festival has roots, solitary or tufted, stems blunt four prism (square), with a groove, densely short pubescence. The Rare grass (tentative name) the seeds have been in Apr. 2, 2011, to pay in Wuhan University, China Center for Type Culture Collection accession, accession number: CCTCC No: P201102 (see attached FIG. 1) The plant has the United States apply for new plant varieties; accepted number: Ser. No. 12/215,016. U.S. Trademark admissibility number: 77633688.

Screening of new drugs by the National Center: Sample ID, nc00168954, including adriamycin (Or substance containing a class of doxorubicin, substance containing a class that has a doxorubicin-resistant) and effective (see Description attached 2). New Drug Screening Center by the National Center: Human cyclooxygenase-2 (COX-2) inhibitory activity reports. Screening of new drugs by the National Center: Antitumor screening by the country's new center: the immune biological activity, T, B lymphocyte proliferation and cytotoxicity assessment results (see Description attached 2). By Hangzhou high-throughput screening of the Center for Drug Screening plant contains seven kinds of active inhibition of cancer (see Description attached 3).

The Department of Agriculture plant varieties have been submitted information materials have been supplemented completed.

The second solution of the present application to complete the task aspect of the invention, a preparation method of the pharmaceutical compositions containing natural human cyclooxygenase (COX-2), a natural doxorubicin (atm) or natural substances class doxorubicin, wherein by the following steps:

(1) Rare grass whole plant cleaned;

(2) The step (1) the whole plant grass gets dean Treasure dehydrated and dried to obtain pulverized powder;

Said step (2) or in one of the following steps of extraction of the herb extract Treasure:

(2) -1 Rare grass decoction, or concentrated, and dried to obtain a powder;

(2) -2 Rare grass decoction, alcohol extract obtained by ethanol extraction, or concentrated, and dried to obtain a powder;

(2) -3 Rare grass or grass decoction of the whole plant. The organic solvent extract obtained by extraction, or a concentrated and dried to obtain a powder;

(2) 4 whole plant or herb extracts Rare grass decoction obtained by supercritical carbon dioxide extraction, or a re-concentrated and dried to obtain a powder;

(2) -5 Rare grass whole plant extract volatile oil;

(3). Powder and I or Volatile oil obtained from Step (2), the step (2) -1, in step (2) -2, in step (2) -3, in step (2)-4 or (2) -5, are mixed with pharmaceutical excipients; and are made forms required;

(4) Sterilization;

(5) Test;

(6) Packaging.

The third solution of the present application to complete the task aspect of the invention: the pharmaceutical compositions containing natural human cyclooxygenase (COX-2), a natural doxorubicin (atm) or natural substances class doxorubicin in the preparation of a medicament for the treatment of nephritis.

The forth solution of the present application to complete the task aspect of the invention: the pharmaceutical compositions containing natural human cyclooxygenase (COX-2), a natural doxorubicin (atm) or natural substances class doxorubicin in the preparation of a medicament for the treatment of cholecystitis and gallbladder polyps.

The fifth solution of the present application to complete the task aspect of the invention: the pharmaceutical compositions containing natural human cyclooxygenase (COX-2), a natural doxorubicin (atm) or natural substances class doxorubicin in the preparation of a medicament in the treatment of tumors.

The sixth solution of the present application to complete the task aspect of the invention: the pharmaceutical compositions containing natural human cyclooxygenase (COX-2), a natural doxorubicin (atm) or natural substances class doxorubicin for the preparation of anti-inflammatory drugs Application.

Nephritis drugs containing the above mentioned natural human cyclooxygenase (COX-2), doxorubicin natural (atm) or a pharmaceutical composition of the material of natural origin for the preparation of doxorubicin, in the preparation of drugs for treating cholecystitis. In preparing cancer drugs, anti-inflammatory medicaments or in the preparation, the mode of administration are: oral tablets, capsules, oral liquid or syrup, oral solution evaporated leavened, topical eye drops, topical ointments, topical wound Yang analgesic ointments, topical inflammation, bleeding, loose bowel, pain powder, does not exclude the injection. Its dose is: Adults measure equivalent material Guizhou Rare grass powder 5 g-6 g daily recommended amount of this application is 5.4 g That is three times daily 1.8 g.

The treatment period (treatment) is seven days. For the treatment of general inflammation requires two courses; treatment of chronic inflammation requires six courses. Treatment of nephritis, cholecystitis, tumors generally require six or more cycles.

The pharmaceutical compositions of the invention can quickly treatment of inflammation, reduce swelling and relieve pain; can also remove viruses, inhibition of tumor cells, and activation of the body immune system activity. Thus, the pharmaceutical compositions of the invention for the treatment of chronic nephritis, acute cholecystitis (including bile reflux cholecystitis), human lymph, lung, liver, stomach, breast, fibroma, leukemia and other cancer diseases. The pharmaceutical compositions of the invention may also be used trachoma, pink eye treatment.

The pharmaceutical composition of the present invention as external use rubbed, can treat bruises, water and fire burns, ulceration sore pus. The pharmaceutical composition of fresh plant material goods, smashed topical present invention can promote healing.

The pharmaceutical compositions of the invention also have a cosmetic effect, can be used to develop cosmetic products.

The following is the drug of the present invention for the preparation of the treatment of nephritis, preparation and treatment of gall bladder polyps Nang inflammatory drugs, or the effect of drug treatment when the tumor was prepared to prove:

Containing native cyclooxygenase (COX-2) doxorubicin pharmaceutical compositions of natural (atm) doxorubicin or a natural substance in the class of drugs for treating nephritis application preparation, nephritis patients diagnosed according to the usual Standard diagnosis; “effective”, “markedly”, “cure” and other standards are in accordance with the traditional standard. Dose of the drug present invention are: adult three times daily 1.8 g. Course of seven days. When six or more cycles, the treatment effect of contrast in the following table:

TABLE 1

| | Effective | Markedly | Cure | Invalid |
|---|---|---|---|---|
| The present invention | 98.3% | 92.5% | 84.2% | |
| drug group | | | | |
| Bunitrolol group | 93.2% | 59% | | |

The present invention drug group

Cure for 7 15 days contains natural human cyclooxygenase (COX-2), when natural doxorubicin atm) or a pharmaceutical composition class doxorubicin natural substances used in the preparation of a medicament for the treatment of cholecystitis, cholecystitis patients diagnosed according to the usual criteria for diagnosis; “effective”, “markedly”, “cure” and other standards are in accordance with the traditional standard. Dose of the drug present invention are: adult three times daily 1.8 g. Course of seven days. When six or more cycles, the treatment effect of contrast in the following table:

TABLE 2

| | Effective | Markedly | Cure | Invalid |
|---|---|---|---|---|
| The present invention | 95.8% | 93.2% | 80% | |
| drug group | | | | |
| Danning tablet group | 82.79% | 78.57% | | 7.14% |

Containing native human cyclooxygenase (COX-2), doxorubicin natural (atm) or natural substance class doxorubicin in the preparation of a pharmaceutical composition of a medicament for the treatment of tumors, cancer (lymphoma) patients diagnosed is the usual criteria for diagnosis; “effective”, “cured”, “cure” and the like in accordance with conventional criteria standards. Dose of the drug present invention are: adult three times a day, every 1.8 go for 7 days. When eight or more cycles, the treatment effect of contrast in the following table:

TABLE 3

| | Effective | Markedly | Cure | Invalid |
|---|---|---|---|---|
| The present invention | 89.3% | 80.5% | 80% | |
| drug group | | | | |
| Paclitaxel group | 48.8% | 32.6% | 16.3% | 2.3% |

Containing native human cyclooxygenase (COX-2), doxorubicin natural (atm) or a pharmaceutical composition of doxorubicin substances of natural origin as a general anti-inflammatory analgesic drugs applied, general inflammation in patients diagnosed according to the usual criteria for diagnosis ; “effective”, “cured”, “cure” and the like in accordance with conventional criteria standards. Dose of the drug present invention are: adult three times daily 1.8 g. Course of seven days When two or more cycles, the treatment effect of contrast in the following table:

TABLE 3

| | Effective | Markedly | Cure | Invalid |
|---|---|---|---|---|
| The present invention | 97.6% | 92.9% | 90% | |
| drug group | | | | |
| Streptomycin group | 14.29% | 78.57% | | 17.14% |

The effect of drug use on the technology of the present invention, please refer to the manual attached sheet 2, 3 and the attached instructions manual attached 4, The experimental high-throughput drug screening report in Hangzhou (description attached 3):

Rare grass provides drug screening, cancer cells, Yu dimensional sarcoma, cervical cancer, liver cancer, four items are strong inhibition; colorectal cancer, prostate cancer, breast cancer, three pulse weak inhibition.

(1) Chinese medicine extracts of A549 cells was significantly drug concentration-dependent inhibition of proliferation of thin tires, the effect was.

(2) Chinese medicine extracts inhibit the breakdown of HT29 cells significantly inhibited cell proliferation, inhibition was drug concentration according to sex.

(3) Chinese medicine extracts details on PC3 cells significantly inhibited cell proliferation, inhibition was concentration-dependent manner.

(4) Chinese medicine extract details on HT1080 cells significantly inhibited cell proliferation, inhibition was drug concentration according to sex.

(5) Chinese medicine extracts details of Hela cells significantly inhibited cell proliferation, inhibition was concentration-dependent manner.

(6) Chinese medicine extract of MDA-MB-231 cells significantly inhibited cell proliferation detail the role of inhibition was concentration-dependent manner.

(7) Chinese medicine extract details on Hepc2 cells significantly inhibited cell proliferation, inhibition was concentration-dependent manner. Results:

Chinese medicine from a dry extract 8 restraint into the concoction 120 grams a concentration ratio of 15, when 55.56 ug/ml, significant cell toxicity, such toxic effects due to the activity of the upper chamber of cell death or long low, migrated to the lower chamber The number of cells is reduced. When the drug concentration is less than 55.56 ug/ml. it has substantially no cellular toxicity. (Experimental test data attached).

The present invention provides a rich human cyclooxygenase (COX-2) and the class of natural or doxorubicin doxorubicin medicinal preparation of a pharmaceutical composition of natural plants, the composition can be achieved using traditional Chinese medicine natural doxorubicin Su (atm) and human cyclooxygenase (COX-2) in the pharmaceutical industry. Avoid the many side effects of synthetic drugs, and has a good therapeutic effect.

### BRIEF DESCRIPTION

FIG. 1 is a traditional Chinese medicine extract inhibited the proliferation of A549 cells in vitro observation of dynamic detection and suppression curve;

FIG. 2 is a traditional Chinese medicine extracts inhibit the proliferation of HT29 cell lines in vitro dynamic detection and suppression effect was observed curve;

FIG. 3 is a traditional Chinese medicine extracts inhibit the proliferation of PC3 cell lines in vitro dynamic detection and suppression effect was observed curve;

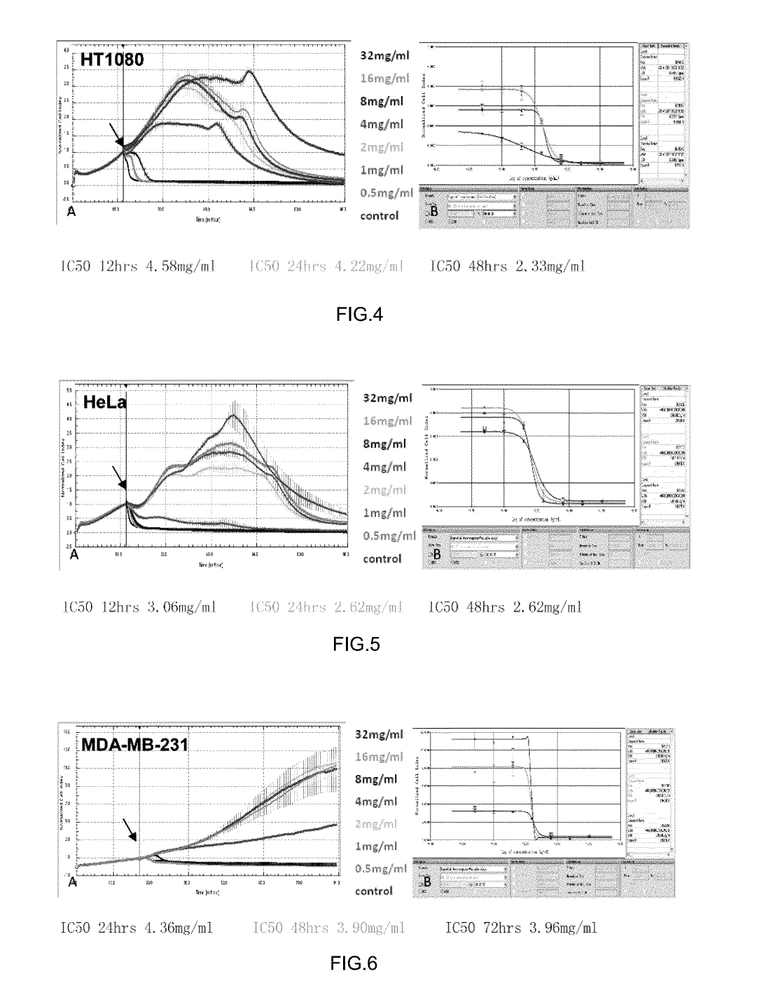

FIG. 4 is a traditional Chinese medicine extracts inhibited HT1080 cell line proliferation dynamic detection and suppression effect was observed curve;

FIG. 5 is a traditional Chinese medicine extracts inhibit the proliferation of Hela cell line motion detection and suppression effect was observed curve;

FIG. 6 is a traditional Chinese medicine extract inhibited MDA-MB-231 cell lines in vitro proliferation of motion detection and suppression effect was observed curve;

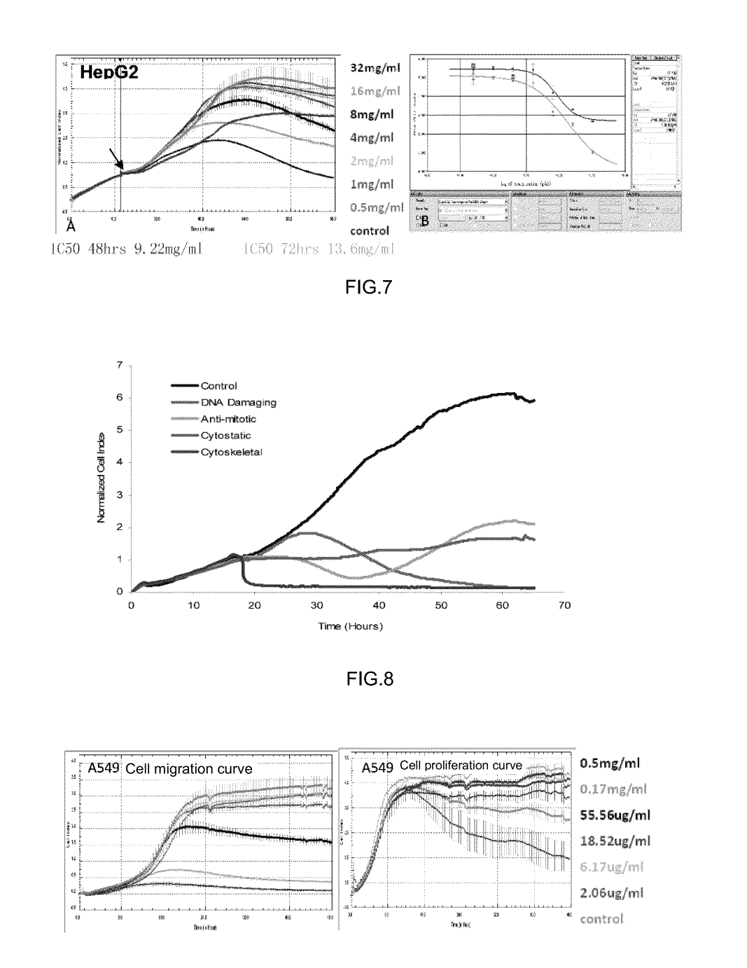

FIG. 7 is a traditional Chinese medicine extract inhibited the proliferation of HepG2 cells in vitro observation of dynamic detection and suppression curve;

FIG. 8 is a compound-specific Atlas;

FIG. 9 is medicine extracts on A549 cells in vitro migration inhibition curve detection capability;

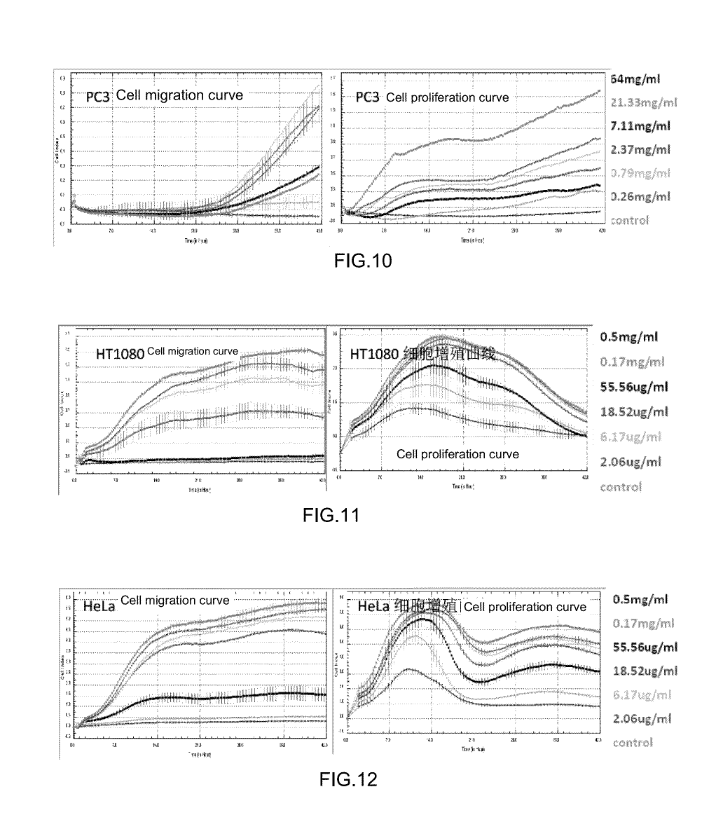

FIG. 10 is a traditional Chinese medicine extracts on inhibition of PC3 cell lines in vitro migration of detection curve;

FIG. 11 is a traditional Chinese medicine extracts of HT1080 cell lines in vitro migration inhibition curve detection capability;

FIG. 12 is a traditional Chinese medicine extracts on HeLa cell lines in vitro migration inhibition curve detection capability;

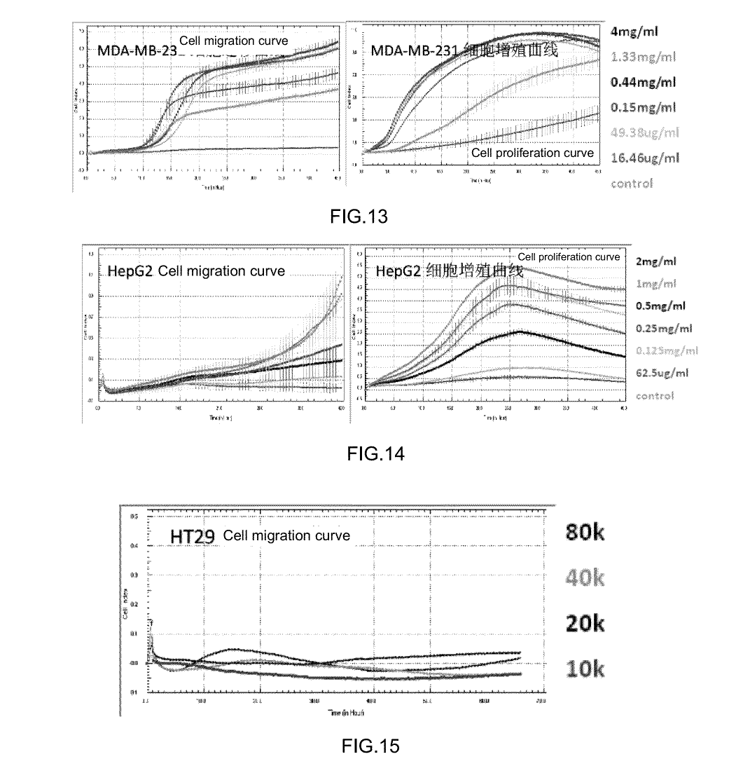

FIG. 13 is a traditional Chinese medicine extracts on MDA-MB-231 cell lines in vitro migration inhibition curve detection capability;

FIG. 14 is a traditional Chinese medicine extracts on HepG2 cell lines in vitro migration inhibition curve detection capability;

FIG. 15 is a HT29 cell lines in vitro migration ability to detect curves;

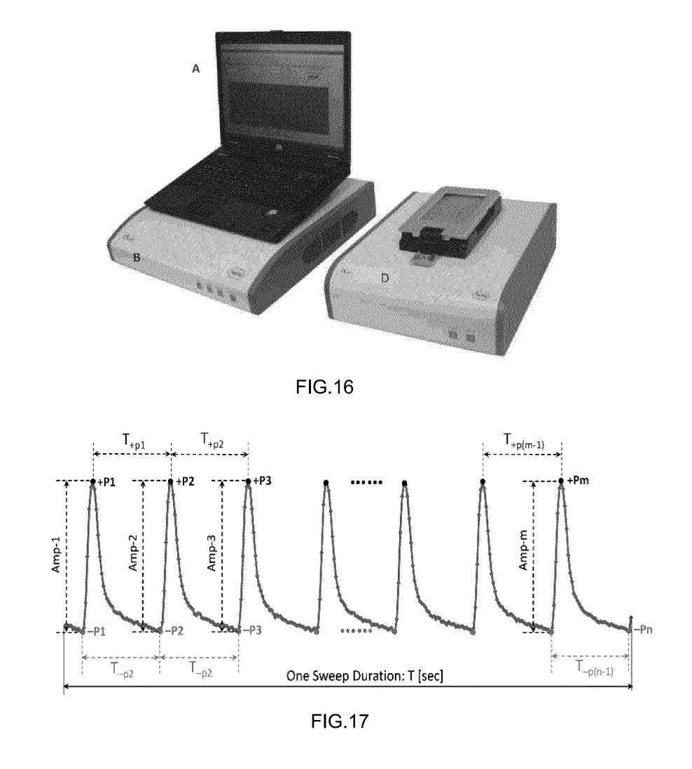

FIG. 16 is a real-time cell analyzer;

FIG. 17, FIG. 18 is a characteristic beating cardiomyocytes and reflected curves were determined state;

FIG. 19 is a cardiac cell model parameters and data analysis page;

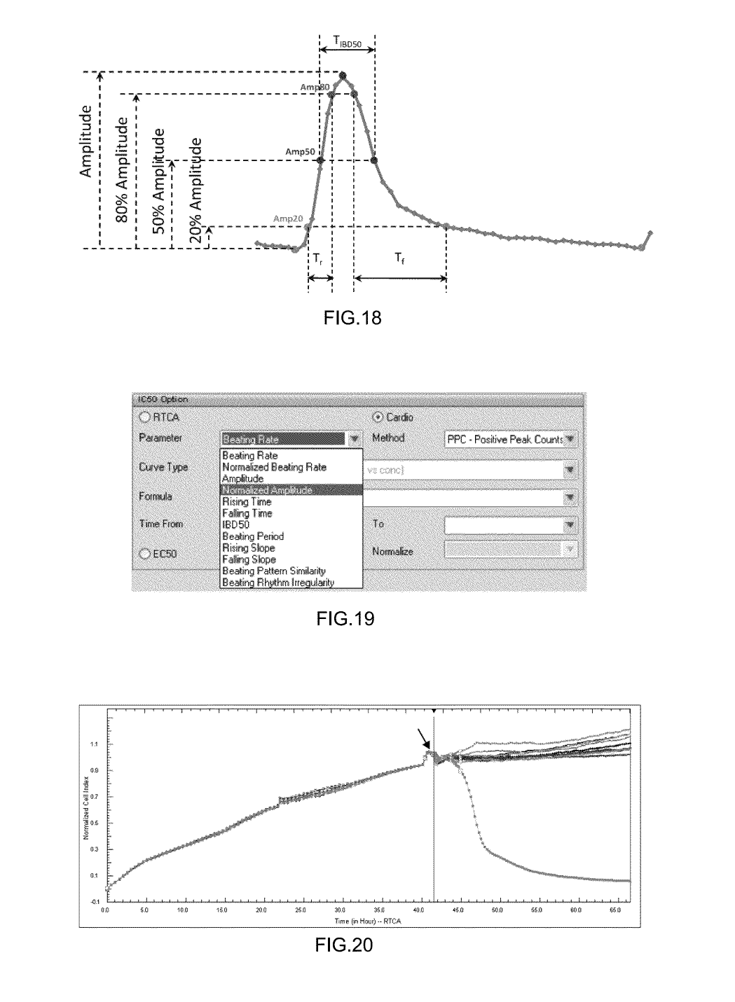

FIG. 20 is a traditional Chinese medicine extract on neonatal rat cardiomyocytes primary activity of the curve;

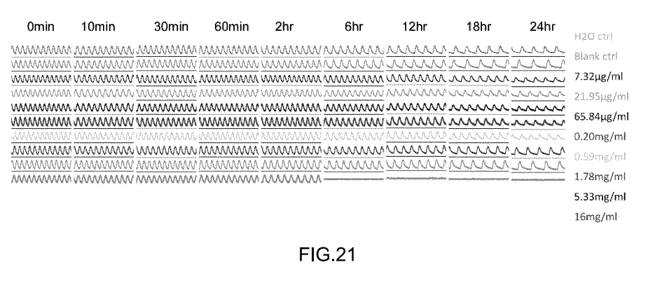

FIG. 21 is a primary cardiomyocytes beat the curve with the time change after dosing;

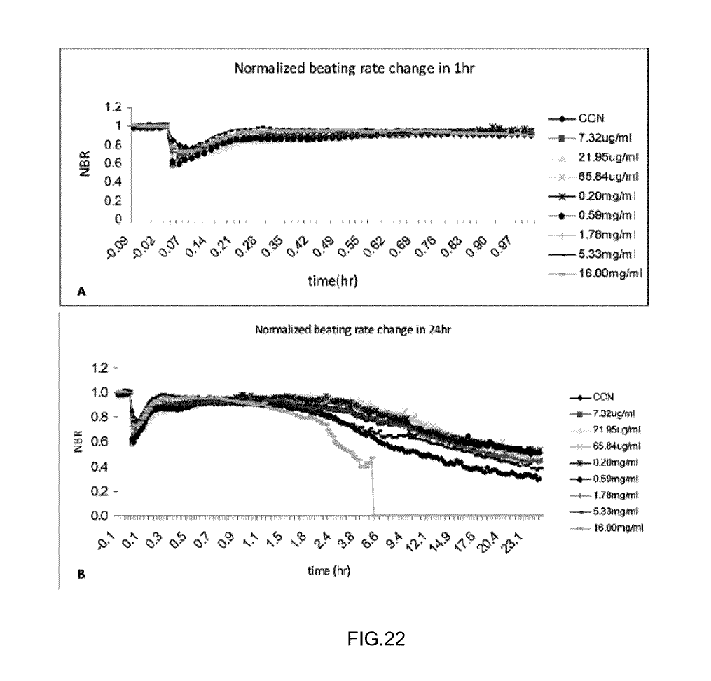

FIG. 22 is a traditional Chinese medicine extract on beating frequency (Beating rate) the role of the curve;

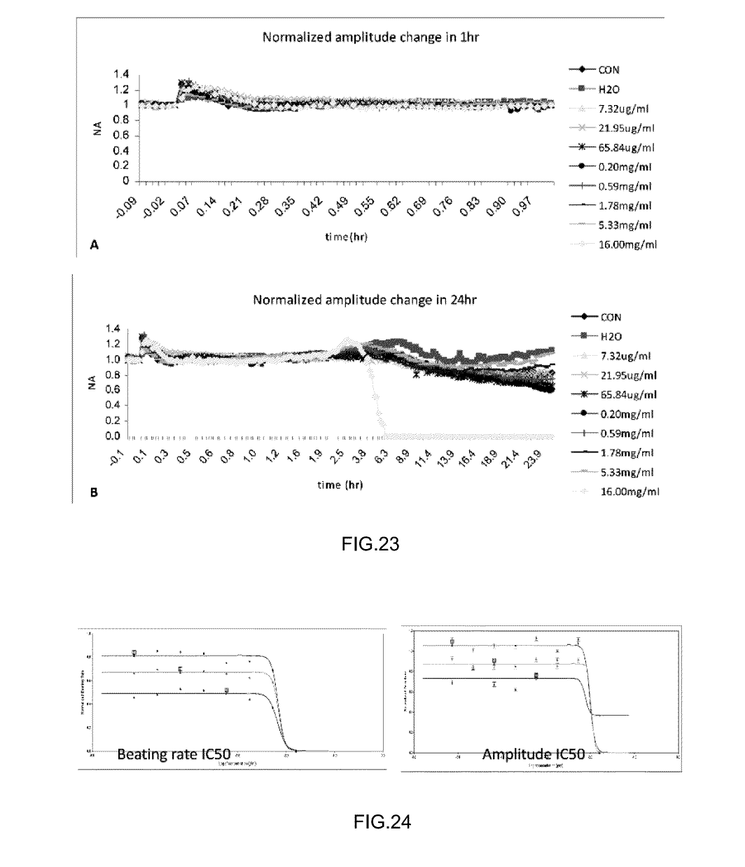

FIG. 23 is a traditional Chinese medicine extract on beating amplitude (Amplitude) the role of the curve;

FIG. 24 is a traditional Chinese medicine extract on IC50 curves neonatal rat cardiomyocytes were beating frequency and amplitude.

### EMBODIMENT

Example 1, containing the natural human cyclooxygenase (COX-2), a natural mixture of the pharmaceutical composition of doxorubicin (atm) or doxorubicin substances of natural origin, and the method for preparing a tablet for clinical use cases.

Natural Adriamycin (doxorubicin or natural class of substances) and human cyclooxygenase pharmaceutical tablet manufacturing method—steaming liquid leavened Method:

Preparations extract the Rare grass: washing, sterilization, with 1 kg of herbs 10 pounds of water ratio, extraction steam distillation to obtain volatile oil and distillates, volatile oil spare, distilled liquid by more than cold for 24 hours, filtered off on a chilly liquid, recovery of ethanol to alcohol-NEB, concentrated into a thick paste, in the dry, crushed, too extract powder, volatile oil and sediment taken evenly sprayed on the extract powder, powder added calcium phosphate unitary, lactose, go light starch, and stearic magnesium and other drugs accessories, made the appropriate dosage forms, ie.

Extract powder tablet production method; dry extract first crushed into fine powder, over 5-6 mesh, wet granulation with ethanol, during tableting. Rotating spray granulation: Join unitary calcium phosphate, lactose, starch, light bulbs, set the dry extract powder coating pan, turn the edges will also wetted with mist sprayed into, gradually wet powder into tablets.

Powder producer method: washing herbs, sterilization, drying system, and dry powder processing microns in particle size 10 um (I-20 um) over 300 mesh sieve, can be made into tablets; Capsule Preparation: 1 kg herbs with water in the ratio of 3 to 10 pounds, Jiaoxiang Method: medicinal treatment: Net system, processing, purification, sterilization, crushed 75 um4.1 over 200 um diameter mesh sieve, filling capsules;

Solvent extraction: Rare grass herbs, properly crushed herbs pounds 10 pounds with a proportion of water, dipping method, percolation, fried by law; optional lipophilic: petroleum ether, benzene,>ether>ethyl acetate. esters>acetone>ethanol>methanol>water (hydrophilic) and the like, in an organic solvent extraction process.

The Treasure of the grass plant preparations in 2006 was sent to Shanghai home Detection Screening Center for Drug Screening detection screening, the results show that the plant contains natural human cyclooxygenase-2_{(cox-2)} activity and immune biological activity, anti-inflammatory treatment have a good effect. Meanwhile lymphatic 1\B cells have a good inhibition, with good prospects.

Attachment-. 1 (save proven a)

Attachment 2 (National Drug Screening proof 3)

Attachment 3 (Hangzhou screening proof 23)

Commissioned services

Based on dynamic real-time label-free xCELLigence cell signaling

(RTCA) technology research and functional analysis system

1. detected by the toxic effects of traditional Chinese medicine extract mediated cell

2. Zhongjun extract cells migrate to detect the impact of shifting

Based on dynamic real-inch xCELLigence unmarked cardiomyocytes Analysis System (RTCA Cardio System) technology

3, Herbal Extracts impact on the detection of myocardial cells

Test report

Customer Name: Xiuying LIN

Report: Hangzhou Qualcomm Center for Drug Screening

Eisen organisms (Hangzhou) Co., Ltd.

Report Date: Feb. 14, 2012 in Hangzhou Qualcomm Biological Drug Screening Center Essen

Hangzhou high Throughput crug screening center

1, detected by the toxic effects of traditional Chinese medicine extract mediated cell

( )Project

The experimental Cell analysis systems using real-time xCELLigence detect inhibition of cell proliferation of Chinese medicine extract and detect drugs that inhibit cell proliferation IC50, using cells A549, HT1 080, Hel, a, MDA-MB-231, HT29, HepG2 and PC3 cells.

( )the experimental apparatus:

In this study, Eisen and Roche jointly developed xCELLigence real-time cell analysis system.

( )Laboratory reagents, supplies:

DMEM medium: Item SH30022 01B, Hyc] one (China).

Modified RPMI-1640 medium: Item SH30809 01B, Hyclone (China).

MEM medium: Item SH30024 01B, Hyclone (China).

F12 medium: Item SH30526 01, Hyclone (China).

MyCOA's 5A medium: Item GNM 16600, Gino (China).

Serum: Item No 16000-044, Gibco (USA).

Trypsin: Item 27250 018, 6ibco (United States).

PBS: Item SH30256 01B, Hyclone (China).

Pipette: Item 4487, Costar.

Centrifuge tube: Item 430791, Corningo

96x E-plate: ACEA.

A549 (non-small cell lung cancer cell line), PC3 (human prostate cancer cell line), HT1080 (human fibroblast sarcoma cell lines), Hela (human cervical carcinoma cell line), MDA MB-231 (human breast cancer cell line), HepG2 (human hepatoma cell line) cells:

ATCCo

Chinese medicine extract provided by Xiuying LIN.

(IV) Drugs Configuration

According to information provided by passenger wide, herbal extract to 20 grams of drugs to get 125 ml concoction, concentration 160 mg/ml.

During the experiment the concentration of the actual needs, diluted with culture medium, added to the cells. Hangzhou Qualcomm Center for Drug Screening Eisen biological

(V) The Design and Test Solutions

Experimental Method—in accordance Layout 96x E-plate cells were seeded, A549 (5000 cells/well), Hela (2500 cells/well), HT1080 (2500 cells/well), MDA-MB-231 (4000 cells/well) I--IT29 (10000 cells / well) HepG2 (10000 cells/well), the PC3 (10000 cells/well) overnight until the cell proliferation, addition of the drug according to the Layout continue detecting 72 h.

Experimental Layout as follows:

Plate 1

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | HT29 | PC3 | MDA-MBA-231 | HepG2 | HeLa | HT1080 |
|---|---|---|---|---|---|---|
| | Chinese medicine extract | | | | | |
| A | 32 mg/ml | 64 mg/ml | 32 mg/ml | 32 mg/ml | 32 mg/ml | 32 mg/ml |
| B | 16 mg/ml | 21.33 mg/ml | 16 mg/ml | 16 mg/ml | 16 mg/ml | 16 mg/ml |
| C | 8 mg/ml | 7.11 mg/ml | 8 mg/ml | 8 mg/ml | 8 mg/ml | 8 mg/ml |
| D | 4 mg/ml | 2.37 mg/ml | 4 mg/ml | 4 mg/ml | 4 mg/ml | 4 mg/ml |
| E | 2 mg/ml | 0.79 mg/ml | 2 mg/ml | 2 mg/ml | 2 mg/ml | 2 mg/ml |
| F | 1 mg/ml | 0.26 mg/ml | 1 mg/ml | 1 mg/ml | 1 mg/ml | 1 mg/ml |
| G | 0.5 mg/ml | 87.79 mg/ml | 0.5 mg/ml | 0.5 mg/ml | 0.5 mg/ml | 0.5 mg/ml |
| H | con | con | con | con | con | con |

Plate2

Plate 2

| | | |
|---|---|---|
| 1 | 2 | 3 |

| | [symbol] | |
|---|---|---|
| | extract | |
| A | 32 mg/ml | |
| B | 16 mg/ml | con |
| C | 8 mg/ml | |
| D | 4 mg/ml | |
| E | 2 mg/ml | |
| F | 1 mg/ml | |
| G | 0.5 mg/ml | |
| H | 0.25 mg/ml | |

[symbol]

indicates data missing or illegible when filed

Diversity signals are set as follows:

| Steps | Cycles | Interval/times | Remarks |
|---|---|---|---|
| Step_l | 1 | 1 min | Measuring base |
| Step_2 | 100 | 15 min | Detection of growth curve |
| Step_3_1 | 200 | 2 min | Detection of drug action |
| Step_3_2 | 300 | 15 min | Detection of drug action |

(VI) Experimental Results

I. RTCA Plot

(1) Chinese medicine extracts inhibit the proliferation of A549 cells in vitro dynamic detection and suppression

(See FIG. 1)

FIG. 1 shows Chinese medicine extracts inhibited A549 cell line proliferation dynamic detection and suppression effect was observed. A is the dynamic map of cell growth and drug effects in real time. Among them, the horizontal axis represents cell proliferation and drug action; vertical axis represents the normalized cell index (NCI). Cell Index prompt cell growth state. The higher its value reflects more living cells. The arrows indicate the dosing time shown in Figure dosing concentration. The graph shows the drug concentration-dependent cells in the role of traditional Chinese medicine extract dynamic response curve, control as a negative control, showed normal cell growth dynamic curve. After B by real-time cell analysis system automatically detects A549 cells calculated by adding the extract of Chinese medicine, the role of drugs in the IC50 values of 12, 24 and 48 hours between IC50-inch point and curve fitting. Seen from the figure, medicine extract on A549 cells significantly inhibited cell proliferation, showing the drug concentration-dependent inhibition.

(2) Chinese medicine extracts inhibit the proliferation of HT29 cell lines in vitro dynamic detection and suppression

(See FIG. 2)

FIG. 2 shows Chinese medicine extracts inhibit the proliferation of HT29 cell lines in vitro inhibitory effect of dynamic testing and observation. A is the dynamic map of cell growth and drug effects in real time. B shows the curve fitting of drug values for 48 and 72 hours time point IC50 and IC50 fitting curve, after HT29 cells automatically detected by Real-Time Cell Analysis System were added to medicine extract. Seen from the figure, medicine extract on HT29 cells significantly inhibited cell proliferation, showing the drug concentration-dependent inhibition.

(3) Chinese medicine extracts inhibit the proliferation of PC3 cell lines in vitro dynamic detection and suppression (See FIG. 3)

FIG. 3 shows Chinese medicine extract inhibited the proliferation of PC3 cell lines in vitro inhibitory effect of dynamic testing and observation. A is the dynamic map of cell growth and drug effects in real time. B shows the curve fitting of drug values for 48 and 72 hours time point IC50 and IC50 fitting curve, after PC3 cells automatically detected by Real-Time Cell Analysis System were added to medicine extract. Seen from the figure, medicine extract on PC3 cells significantly inhibited cell proliferation, showing the drug concentration-dependent inhibition.

(4) Chinese medicine extracts inhibited HT1080 cell line proliferation dynamic detection and suppression

(See FIG. 4)

FIG. 4 shows Chinese shows medicine extracts inhibited HT1080 cell line proliferation dynamic detection and suppression effect was observed. A is the dynamic map of cell growth and drug effects in real time. B shows the curve fitting of drug values for 48 and 72 hours time point IC50 and IC50 fitting curve, after HT1080 cells automatically detected by Real-Time Cell Analysis System were added to medicine extract. Seen from the figure, medicine extract on HT1080 cells significantly inhibited cell proliferation, showing the drug concentration-dependent inhibition.

(5) Chinese medicine extracts inhibit the proliferation of HeLa cells in vitro dynamic detection and suppression

(See FIG. 5)

FIG. 5 shows medicine extract inhibited Hela the proliferation of cell lines in vitro inhibitory effect dynamic testing and observation. A is the dynamic map of cell growth and drug effects in real time. B shows the curve fitting of drug values for 48 and 72 hours time point IC50 and IC50 fitting curve, after Hela cells automatically detected by Real-Time Cell Analysis System were added to medicine extract. Seen from the figure, medicine extract on Hela cells significantly inhibited cell proliferation, showing the drug concentration-dependent inhibition.

(6) Chinese medicine extracts inhibited MDA-MB-231 cell lines in vitro proliferation of motion detection and suppression

(See FIG. 6)

FIG. 6 shows Chinese medicine extract inhibited MDA-MB-231 cell line proliferation dynamic detection and suppression effect was observed. A is the dynamic map of cell growth and drug effects in real time. B shows the curve fitting of drug values for 48 and 72 hours time point IC50 and IC50 fitting curve, after MDA-MB-231 cells automatically detected by Real-Time Cell Analysis System were added to medicine extract. Seen from the figure, medicine extract on MDA-MB-231 cells significantly inhibited cell proliferation, showing the drug concentration-dependent inhibition.

(7) Chinese medicine extracts inhibited the proliferation of HepG2 cells in vitro dynamic detection and suppression

(See FIG. 7)

FIG. 7 shows Chinese medicine extracts inhibited the proliferation of HepG2 cells in vitro inhibitory effect of dynamic testing and observation. A is the dynamic map of cell growth and drug effects in real time. B shows the curve fitting of drug values for 48 and 72 hours time point IC50 and IC50 fitting curve, after HepG2 cells automatically detected by Real-Time Cell Analysis System were added to medicine extract. Seen from the figure, medicine extract on HepG2 cells significantly inhibited cell proliferation, showing the drug concentration-dependent inhibition.

2 Chinese medicines extracts on cell lines cytotoxicity

| | IC50 (mg/ml) | | | |
|---|---|---|---|---|
| Cell | 12 hrs | 24 hrs | 48 hrs | 72 hrs |
| A549 (5K/well) | 2.92 | 2.09 | 2.83 | / |
| HT29 (10K/well) | / | / | 22.6 | 8.65 |
| PC3 (10K/well) | / | 7.68 | 7.57 | 7.68 |
| HT1080 (2.5K/well) | 4.58 | 4.22 | 2.33 | / |
| HeLa (2.5K/well) | 3.06 | 2.62 | 2.62 | / |
| MDA-MB-231 (4K/well) | / | 4.36 | 3.90 | 3.96 |
| HepG2 (10K/well) | / | / | 9.22 | 13.6 |

(Vii) The conclusions of the experiment and predict possible problems and suggestions

1. An experimental test of the traditional Chinese medicine extracts on human non-small cell lung cancer cell line (A549), human colon cancer cell line (HT29), human prostate cell lines (PC3), human fibroblast sarcoma cell lines (HT1080), human cervical cancer cell lines (Hela), human breast cancer cell line (MDA-MB-231), 7 poison role of human hepatoma cell line (HepG2) cells in different organs cell lines.

2. due to the role of drugs in different situations in different cell lines, select four time points (after dosing 12 hr, 24 hr, 48 hr, 72 hr), according to the specific situation of individual cells, the IC50 values obtained corresponding point in time.

3 .Chinese medicine extracts on selected seven inhibited the proliferation of cell lines, showing good concentration gradient, in which the HT1080, HeLa, MDA-MB-231, A549 cells stronger effect on HepG.2, HT29, PC3 cells weaker effect.

4.from a drug development point of view, can continue to study the drug therapy of cancer in cancer, cervical cancer, breast cancer, and can be considered experimental drugs on non-tumor cells of the cell line if there cytotoxicity.

The compound mechanism 5 Discussion: A compound-specific response patterns, herbal extracts in these cell lines exhibited significant cytotoxicity in H29 and Atlas PC3 cells showed cytotoxic effects characteristic DNA Damage.

(See FIG. 8)

FIG. 8 compound-specific maps. Impedance data obtained from a real system to give compound xCELLigence specific pattern, the biological mechanism of action of these compounds relies on the map. In this example, the biological compound with different target specificity and mechanism of action, the compounds can produce a special pattern. For compounds having specific effects of these maps can be used to predict some of the assumptions set verifiable manner. Experimental record information Stored in Essen biological (Hangzhou) Co., Ltd. customer database.

Code: 1112051511P3, 1112081603P3, 1112141440P5, 1112201057P5

2. Detection medicine extract on cell migration of

(I) Project

In this study, xCELLigence real-time cell analysis system to detect inhibition of a traditional Chinese medicine extracts provided by Guizhou herbs on cell migration, using cells A549, PC3, HT1080, Hela, MDA-MB-23 HepG2

(II) Laboratory Instruments

In this study, Eisen and Roche jointly developed xCELLigence DP real-time cell analysis system.

(III) Test Reagents, Consumables

DMEM medium: Item SI-130022.08B, Hyclone (China).

Modified RPMI-1640 medium: Item SH30809.01B, Hyclone (China).

MEM medium: Item SH30024.01B, Hyclone (China).

F12 medium: Item SH30526,01, Hyclone (China). Serum: Item No. 16000-044, Gibco (USA),

trypsin: Item No. 27250-018, Gibco (USA).

DMSO: Item No. D2650, Sigma (USA).

PBS: kern SH30256,01B, Hyclone (China

Pipette: Item 4487, Costar.

Centrifuge tube: Item 430791, Corning.

96x E-plate: ACEA

CIM plate: ACEA

A549 (non-small cell lung cancer cell line), PC3 (human prostate cell lines), HT1080 (human fibroblast sarcoma cell lines), Hela (human cervical carcinoma cell line), MDA-MB-231 (human breast cancer cell line), HepG2 (human hepatoma cell line) cells: ATCC.

Chinese medicine extract: Xiuying LIN

(IV) Drug Preparation Methods

According to information provided by the customer, herbal extract 20 g 125 ml concoction of drugs to get a concentration of 160 mg/ml.

During the experiment the concentration of the actual needs, diluted with the culture medium.

(V) Experimental Design and Solutions

Methods:

In accordance with the CIM plate Layout room added serum-free medium containing 10% of the drug formulation in the chamber with the serum-free medium was added to the drug formulation, at the DP system detects the baseline. Serum-free medium was then added and the suspension has been re-blind 1 h incubation of the cells with the compound in the chamber, on the DP detection 24 h-48 h.

At the same time, the control cells proliferation was set parallel to the 96x E-plate. According Layout medium containing 10% serum formulated drugs on SP detection baseline. Then added with 10% and resuspended in serum-free medium has been incubated with the compound 1 h cells detected in the SP 24 h-48 h.

Cell concentration used: A549 (60 k/well), PC3 (40 k/well), HT1080 (30 k/well), Hela (30 k/well), MDA-MB-231 (80 k/well), HepG2 (80 k/well)

Experimental Layout as follows:

| | | | | Cell proliferation | |
|---|---|---|---|---|---|
| | | Cell migration | | control | |
| | | Line1 | Line2 | Line1 | Line2 |
| | | A549 60k/well | | A549 60k/well | |
| A | Chinese | 0.5 mg/ml | | 0.5 mg/ml | |
| B | medicine | 0.17 mg/ml | | 0.17 mg/ml | |
| C | extract | 55.56 ug/ml | | 55.56 ug/ml | |
| D | | 18.52 ug/ml | | 18.52 ug/ml | |
| E | | 6.17 ug/ml | | 6.17 ug/ml | |
| F | | 2.06 ug/ml | | 2.06 ug/ml | |
| G | Control | CON | | CON | |
| H | Serum free | SF CON | | SF CON | |
| | | Cell migration | | Cell proliferation control | |
| | | Line1 | Line2 | Line1 | Line2 |
| DP2 | | PC3 40k/well | | PC3 40k/well | |
| A | Chinese | 64 mg/ml | | 64 mg/ml | |
| B | medicine | 21.33 mg/ml | | 21.33 mg/ml | |
| C | extract | 7.11 mg/ml | | 7.11 mg/ml | |
| D | | 2.37 mg/ml | | 2.37 mg/ml | |
| E | | 0.79 mg/ml | | 0.79 mg/ml | |
| F | | 0.26 mg/ml | | 0.26 mg/ml | |
| G | Control | CON | | CON | |
| H | Serum free | SF CON | | SF CON | |
| | | Cell migration | | Cell proliferation control | |
| | | Line1 | Line2 | Line1 | Line2 |
| DP3 | | HT1080 30k/well | | HT1080 30k/well | |
| A | Chinese | 0.5 mg/ml | | 0.5 mg/ml | |
| B | medicine | 0.17 mg/ml | | 0.17 mg/ml | |
| C | extract | 55.56 ug/ml | | 55.56 ug/ml | |
| D | | 18.52 ug/ml | | 18.52 ug/ml | |
| E | | 6.17 ug/ml | | 6.17 ug/ml | |
| F | | 2.06 ug/ml | | 2.06 ug/ml | |
| G | Control | CON | | CON | |
| H | Serum free | SF CON | | SF CON | |
| | | Cell migration | | Cell proliferation control | |
| | | Line1 | Line2 | Line1 | Line2 |
| DP4 | | Hela 30k/well | | Hela 30k/well | |
| A | Chinese | 0.5 mg/ml | | 0.5 mg/ml | |
| B | medicine | 0.17 mg/ml | | 0.17 mg/ml | |
| C | extract | 55.56 ug/ml | | 55.56 ug/ml | |
| D | | 18.52 ug/ml | | 18.52 ug/ml | |
| E | | 6.17 ug/ml | | 6.17 ug/ml | |
| F | | 2.06 ug/ml | | 2.06 ug/ml | |
| G | Control | CON | | CON | |
| H | Serum free | SF CON | | SF CON | |
| | | Cell migration | | Cell proliferation control | |
| | | Line1 | Line2 | Line1 | Line2 |
| | | MDA-MB-231 80k/well | | MDA-MB-231 80k/well | |
| A | Chinese | 4 mg/ml | | 4 mg/ml | |
| B | medicine | 1.33 mg/ml | | 1.33 mg/ml | |
| C | extract | 0.44 mg/ml | | 0.44 mg/ml | |
| D | | 0.15 mg/ml | | 0.15 mg/ml | |
| E | | 49.38 ug/ml | | 49.38 ug/ml | |
| F | | 16.46 ug/ml | | 16.46 ug/ml | |
| | | Cell migration | | Cell proliferation control | |
| | | Line1 | Line2 | Line1 | Line2 |

| | | HepG2 80k/well | HepG2 80k/well |
|---|---|---|---|
| A | Chinese | 2 mg/ml | 2 mg/ml |
| B | medicine | 1 mg/ml | 1 mg/ml |
| C | extract | 0.5 mg/ml | 0.5 mg/ml |
| D | | 0.25 mg/ml | 0.25 mg/ml |
| E | | 0.125 mg/ml | 0.125 mg/ml |
| F | | 62.5 ug/ml | 62.5 ug/ml |
| G | Control | CON | CON |
| H | Serum free | SF CON | SF CON |

Signal acquisition is set as follows:

| Steps | Cycles | Interval/times | Remarks |
|---|---|---|---|
| Step_1 | 1 | 1 min | Measuring base |
| Step_2 | 200 | 15 min | Detection of cell migration under the |
| | | | curve detection of drugs |

The experimental results

I.RTCA Plot

(1) Chinese medicine extracts to detect inhibition of A549 cell lines in vitro migration capabilities.

(See FIG. 9)

FIG. 9 shows Chinese medicine extracts to detect inhibition of A549 cell lines in vitro migration capabilities.

Left curve drugs that inhibit cell migration, drug right is carded out in parallel on cell proliferation of real-time dynamic map. Among them, the horizontal axis represents cell culture and drug action time; vertical axis represents the normalized cell index (NCI). Cell Index prompt cell growth state. In the cell migration assay, the higher the value, the number of cells migrated to the reaction chamber, the more; experiments in Cell proliferation, the higher the value the more the reflection of the living cells. As shown in Figure dosing concentration.

Left shows the cellular response curve in the role of traditional Chinese medicine extract under the drug concentration-dependent cell migration ability, Control as a negative control, display abnormal cell migration dynamic curve.

On the right is a cell in the role. of traditional Chinese medicine extract drug concentration-dependent cell proliferation, Control as a negative control, showing normal fine.

When the drug concentration is higher than the added 55.56 ug/ml, significant cell toxicity, the toxicity of this chamber due to cell death or the activity decreased, the number of cells migrated to the lower chamber is reduced. When the drug concentration is below 55.56 ug/ml, basically no toxic effects on cells, cell migration, but also subject to certain inhibition, and when the drug was concentration-dependent inhibition, so the drugs on the A549 has some inhibition of cell migration effect.

(2) In vitro migration of traditional Chinese medicine extracts on inhibition of PC3 cell line detection capability,

(See FIG. 10)

FIG. 10 shows Chinese medicine extract inhibition detection of PC3 cell lines in vitro migration capabilities. Left curve drugs that inhibit cell migration, drug right is carried out in parallel on cell proliferation of real-time dynamic map. As shown in Figure dosing concentration.

Left shows the cellular response curve in the role of traditional Chinese medicine extract under the drug concentration-dependent cell migration ability, Control as a negative control, display abnormal cell migration dynamic curve.

The right is the role of cell extracts in medicine under the drug concentration-dependent inhibition of cell proliferation, Control as a negative control, showed normal cell proliferation dynamic curve.

This experiment demonstrates the different concentration cell extract of cells significantly toxic effect on the toxic effects due to the lower chamber or the active cell death, the number of cells migrated to the lower chamber is reduced. But in traditional Chinese medicine extract concentrations below 7.11 mg/ml, under the action of the drug, cell migration was higher than control, stimulate migration.

(3) inhibition of detection of traditional Chinese medicine extract HT1080 cell lines in vitro migration capabilities.

(See FIG. 11)

FIG. 11 shows inhibition of detection of traditional Chinese medicine extract HT1080 cell lines in vitro migration capabilities.

Left curve drugs that inhibit cell migration, drug right is carried out in parallel on cell proliferation of real-time dynamic map. As shown in Figure dosing concentration.

Left shows the cellular response curve in the role of traditional Chinese medicine extract under the drug concentration-dependent cell migration ability, Control as a negative control, display abnormal cell migration dynamic curve.

The right is the role of Cell extracts in medicine under the drug concentration-dependent inhibition of cell proliferation, Control as a negative control, showed normal cell proliferation dynamic curve. When the drug concentration is higher than the added 18.52 ug/ml, significant cell toxicity, the toxicity of this chamber due to cell death or the activity decreased, the number of cells migrated to the lower chamber is reduced. When the drug concentration is less than or equal to 18.52 ug/ml, basically no toxic effects on cells, cell migration, but also subject to certain inhibition, and when the drug was concentration-dependent inhibition, so that the drug has some migration of A549 cells inhibition.

(4) Chinese medicine extracts to detect inhibition of HeLa cell lines in vitro migration capabilities.

(See FIG. 12)

FIG. 12 shows Chinese medicine extracts to detect inhibition of HeLa cell lines in vitro migration capabilities. Left curve drugs that inhibit cell migration, drug right is carried out in parallel on cell proliferation of real-time dynamic map. As shown in Figure dosing concentration.

Left shows the cellular response curve in the role of traditional Chinese medicine extract under the drug concentration-dependent cell migration ability, Control as a negative control, display abnormal cell migration dynamic curve.

The right is the role of cell extracts in medicine under the drug concentration-dependent inhibition of cell proliferation, Control as a negative control, showed normal cell proliferation dynamic curve.

Drug was added to the cells significantly toxic effect on the toxic effects due to the lower chamber or the active cell death, the number of cells migrated to the lower chamber is reduced. The drug had no significant inhibition of cell migration.

(5) Chinese medicine extract inhibition detection MDA-MB-231 cell lines in vitro migration capabilities.

(See FIG. 13)

FIG. 13 shows Inhibition of detection of traditional Chinese medicine extract MDA-MB-231 cell lines in vitro migration capabilities. Left curve drugs that inhibit cell migration, drug right is carried out in parallel on cell proliferation of real-time dynamic map. As shown in Figure dosing concentration.

Left shows the cellular response curve in the role of traditional Chinese medicine extract under the drug concentration-dependent cell migration ability, Control as a negative control, display abnormal cell migration dynamic curve.

The right is the role of cell extracts in medicine under the drug concentration-dependent inhibition of cell proliferation, Control as a negative control, showed normal cell proliferation dynamic curve.

Early in the experiment (pre-20 hr), the drug was added to the migrated cells MDA-MB-231 has a stimulating effect. And when the drug concentration is less than 0.15 mg/ml, no apparent cell toxicity at this time, the higher the amount of drug added, the stronger stimulation of cell migration. When adding the drug concentration is higher than 0.4 mg/ml, cells are more obvious toxic effects, such toxic effects on the chamber due to cell death or decreased activity, the number of cells migrated to the lower chamber decreased.

(6) Chinese medicine extracts of HepG2 cells detect inhibition in vitro migration capabilities.

(See FIG. 14)

FIG. 14 shows Chinese medicine extract inhibition detection of HepG2 cells in vitro migration capabilities. Left curve drugs that inhibit cell migration, drug right is carried out in parallel on cell proliferation of real-time dynamic map. As shown in Figure dosing concentration.

Hangzhou Qualcomm Center for Drug Screening Eisen biological

Hangzhou high Throughput crug screening center left shows the cellular response curve in the role of traditional Chinese medicine extract under the drug concentration-dependent cell migration ability, Control as a negative control, display abnormal cell migration dynamic curve.

The right is the role of cell extracts in medicine under the drug concentration-dependent inhibition of cell proliferation, Control as a negative control, showed normal cell proliferation dynamic curve.

Drug was added to the cells significantly toxic effect on the toxic effects due to the lower chamber or the active cell death, the number of cells migrated to the lower chamber is reduced. That the drug had no significant inhibition of cell migration.

(7) HT29 cell lines in vitro migration testing.

(See FIG. 15)

FIG. 15 shows the ability to detect in vitro migration FIG. 15 HT29 cells. As shown, the cell concentrations were 80 k/well, 40 k/well, 20 k/well, when 10 k/well, HT29 no apparent cat migration, and therefore can not detect drug inhibition of cell migration.

(Vii) the conclusions of the experiment and predict possible problems and suggestions

(1) An experimental test of the traditional Chinese medicine extracts on human non-small cell lung cancer cell line (A549), human colon cancer cell line (HT29), human prostate cell lines (PC3), human fibroblast sarcoma cell lines (HT1080), human cervical cancer cell line (HeLa), human breast cancer cell line (MDA-MB-231), 7 different organ cell lines in vitro inhibition of migration of human hepatoma cell line (HepG2), while parallel detection influence of drugs on cell proliferation, The purpose is to detect cell proliferation inhibition detect drugs on cell migration was not caused by cytotoxicity,

(2) high concentrations of Chinese medicine extracts on A549 and HT1080 cells have significant toxic effects, such toxic effects on the chamber due to cell death or decreased activity, the number of cells migrated to the lower chamber decreased. While low concentrations of drugs on cell migration has a certain extent.

3. Chinese medicine extracts of HeLa and HepG2 cells have significant toxic effects, such toxic effects on the chamber due to cell death or decreased activity, the number of cells migrated to the lower chamber decreased. While low concentrations of the drug had no significant inhibition of cell migration, The drug had no inhibitory effect on cell migration.

4. Herbal extract high concentrations of PC3 and MDA-MB-231 cells have significant toxic effects, such toxic effects on the chamber due to cell death or decreased activity, the number of cells migrated to the lower chamber decreased, While low concentrations of drugs on cell migration have a stimulating effect. Experimental record information

Stored in Essen biological (Hangzhou) Co., Ltd, customer database.

Code: 1112211551012, 1112211605P4, 1112271558012, 1112271559P4, 1112311454P6, 1112311452012, 1201101537012, 1201101540P2, 1202021535012, 1202021539P1

4. herbal extracts affect the detection of myocardial cells

1. Project

The experiments using real-time myocardial cell analysis system, the primary neonatal rat cardiomyocytes, herbal medicine offers Guizhou extract drug cardiac safety testing and evaluation. Evaluation parameters used in this experiment was time-dependent in vitro cardiomyocyte beating frequency, amplitude and beating cardiomyocytes rhythmic beating cardiomyocytes and wave.

2. Background

2.1 Background

Eisen creature is a commitment to the development and manufacturing of the company's own patented technology with impedance-based real-time cell detector tech biological company. Eisen company microelectrode sensor is mounted on the bottom of the innovative design of the microplate can be used for real-time label-free detection of the state of cells, with high throughput, high accuracy, high sensitivity detection and quantitative advantages.

Heart disease or heart function by the cardiac toxicity of drug-induced damage to threaten human health and safety. Further reveal significant heart disease mechanisms and to explore new and effective prevention and control measures on this basis, as well as in drug development process, revealing some of the drugs cardiotoxicity heart researchers faced major challenges workers. Eisen biological and Roche to jointly develop real-time myocardial cell analysis system (xCELLigence RTCACardio System), can dynamically monitor cardiomyocytes excitation-contraction coupling reaction, and real-time short-range long-range detection and record beating cardiomyocytes signals. When the compound effects on myocardial cells affect cell morphology and activity, or cause the associated channel openness to change, or cause shrinkage excited conductivity changes can be monitored in real time by real-time myocardial cell analysis system. The system can be used to screen for myocardial cell contraction excitability changes in conductivity drugs, including ion channel and non-ion channel drug targets. Eisen companies use embryonic stem cells in myocardial cells (CorAt, Axiogenesis) and human iPS cells induced cardiac cells to more than 50 kinds of known compounds were verified, and later made additional verification with primary rat cardiomyocytes. The results show that real-time myocardial cell analysis system can reliably and quantitative detection of arrhythmia compounds as well as the role of non-ERG channels and voltage-gated calcium channel compounds on cardiac cells. Therefore, the system can be used as an early screening cardiotoxicity during drug development tool compounds, drug screening and toxicity related to study drug for high throughput.

Work:

The goal of this project is to use the impedance detection system, the ability to beat the rate of primary cells myocardial drug cardiac safety testing and assessment, evaluation and identification of compounds caused. Each of the projects using seven different concentrations of the compounds tested at different time points before and after the detection of the selected monitor cell beating state, by observing a dose-dependent effect of the extract of Chinese medicine in primary rat cardiomyocytes to evaluate its primary beating cardiomyocytes influence.

2.2 System Introduction

Real-time myocardial cell analysis system (xCELLigence RTCA Cardio System) continuation of the existing biological Eisen independent patent rights based microelectronics impedance detection system cells (including RTCA SP, MP, DP system) function can be used to detect myocardial functional beating cells and cardiomyocytes for drug toxicity testing. The core of the system is to be integrated into the display microelectronic sensor cells 96-well plates

(Cardio-Plate 96) at the bottom, it greatly improves the data acquisition rate, RTCA myocardial cell analysis system in addition to having similar RTCA SP, MP and DP systems for a long time to detect the function of cell growth and proliferation, but also excited to be able to detect myocardial cells contraction coupling forming cells beating.

RTCA myocardial cell analysis system consists of the following parts (see figure below):

A: real-time cell analysis system console (RTCA Cardio Control Unit)

B: real-time cell analyzer (RTCACardio Analyzer)

C: cell test bench (RTCACardio Station)

D: detection board (Cardio-Plate %)

(See FIG. 16)

The cells were seeded into 96-well assay plates in myocardial cells, the cells attached to the surface of the sensor electronics. RTCA test bench on carbon dioxide cell incubator (5% CO2, 37° C.), the detection of myocardial cell plate in RTCA testing work bench, and with analyzers and real-time cell analysis system console is turned on. The sensor surface can be detected in real time by detecting the electrical impedance of the table and the signal analyzer, the measured electrical impedance of the signal in real time by the cell analysis system RTCA Cardio console and software for analysis, quantitative real-time to provide the biological state of the cell, including cell count, motility, morphology, and cytoskeletal dynamics and other information. Primary rat primary cardiomyocytes myocardial cell model that can be used to study many cardiac cell morphology, biochemical, electrophysiological and pharmacological aspects. The model is a proven way to study for drug transport, toxicity, and electrophysiological characteristics. The rats were 10 and 75% ethanol disinfection newborn within 24 h after sternal edge into the shears, scissors avoid breaking the digestive tract to prevent contamination. Sterile clip ventricular site carefully atrium and aorta of glass quickly placed in ice-cold serum-free DMEM, repeatedly washed 3 times, wash the remaining blood cells, the size of the tissue cut 0.5 mm×0.5 mm×0.5 mm×block, into a small glass bottle was added 0.07% trypsin and 0.07% collagenase 1 digestion 1.5 ml of supernatant was collected after digestion, add appropriate amount (equal volume of the supernatant) containing 10% fetal bovine serum DMEM medium termination digestive enzymes, repeat the above steps until the tissue digestion disappear. The pore size of 200 mesh sieve to filter the undigested tissue, and the supernatant was centrifuged 5 min (800 r/min), discard the supernatant, DMEM medium containing 10% fetal bovine serum disperse precipitated cells were seeded in culture flasks, Carbon dioxide cell incubator (5% CO2, 37° C.) in stationary culture 60 min (differential adhesion), to collect non-adherent cells, its density 15,000 cells per well were seeded to gelatin-treated 96-well plates in the detection of myocardial cells, placed Cardio were detected on the system.

3. Experimental design and program

4.

3. Shang experimental apparatus:

The experiments using real-time myocardial cell analysis system Eisen and Roche jointly developed.

| Product Name | Cat.No. | Description |
|---|---|---|
| RTCA Cardio Station | 6417019001 | RTCA Cardio Station for |
| | | screening of cardiomyocytes |
| RTCA Cardio Analyzer | 6416993001 | RTCA HT Analyzer ( incl.Power |
| | | Cable and Serial Cable ) |
| RTCA Cardio | 6200184001 | Basic Notebook HP 6930p, pre- |
| Control Unit | | installed RTCA Cardio Software |
| E-Plate Cardio 96 | 6417051001 | 1 × 6 plates |
| E-Plate Cardio 96 | 6417035001 | 6 × 6 plates |

3.2 Reagents, consumables:

DMEM medium: Item 3H30022.08B, Hyclone (China).

Serum: Item No. 16000-044, Gibe( )(USA).

Trypsin: Item No. 27250-018, Gibco (USA).

Collagenase: Item No. 17101-015, Gibco (USA).

DMSO: Item No. D2650, Sigma (USA).

HBSS solution: 14175, Gibco (USA).

Gelatin: item 48732, Sigma (USA).

PBS: Item SH30256.01B, Hyclone (China). 3.3 The method of preparation of drugs

According to information provided by the customer, herbal extract 20 g 125 ml concoction of drugs to get a concentration of 160 mg/ml, During the experiment the concentration of the actual needs, diluted with culture medium, added to the cells.

3.4 Experimental design and program of primary cultured neonatal rat cardiomyocytes, seeded 17,000 cells per well, placed in real-time myocardial cells after vaccination analysis system for 48 hours according to the following steps and parameter detection (see signal acquisition set the table), and in After the exchange of medium, medium was changed two hours until the cell signal stability, according to the concentration of the compound added to the cells corresponding hole. After dosing the cells reset detection system for real-time acquisition cardiomyocytes beat signal. Each compound is prepared using two or more wells.

| | | C2 | C3 | C4 | C5 | C6 | C7 | C8 | C9 | C10 | C11 |
|---|---|---|---|---|---|---|---|---|---|---|---|
| A | | | | | | | | | | | |
| B | Chinese medicine | 7.32 | 21.95 | 65.84 | 0.20 | 0.59 | 1.78 | 5.33 | 5.33 | 16.00 | CON |
| C | extract | μg/ml | μg/ml | μg/ml | mg/ml | mg/ml | mg/ml | mg/ml | mg/ml | mg/ml | |

Acquired signals are set as follows:

| Steps | Cycles | Interval/times | Monitoring time/times | Remarks |
|---|---|---|---|---|
| 0 | 10 | 1 min | 20 seconds | Measuring base |
| 1 | 48 | 15 min | 20 seconds | Adherent cell |
| | | | | and growth |
| 2 | 8 | 1 min | 20 seconds | Replacing liquid |
| 3 | 10 | 1 min | 20 seconds | Before dosing |
| 4 | 60 | 1 min | 20 seconds | After dosing |

3.5 Data Analysis

3.5.1 Inch between each point in the calculation cell beating frequency (beating rate), and draw the curve beating (beating rate)

3.5.2 Analysis parameters include:

Time-dependent parameters beat frequency Time dependent beating rate
Standardized beat frequency Normalized beating rate
Time-dependent jitter amplitude Time dependent amplitude
Standardized bouncing Normalized amplitude
increase the overall activity of the drug before and after the state of the cell Cell Index
Time-dependent beating rhythm abnormalities Time dependent beating rhythm irregularity
Time-dependent phase 4 beats at a rate Time dependent beating similarity analysis parameters defined

Each cell corresponds to a cardiomyocyte beating muscle cells excitation-contraction coupling. The order of appearance of the pulse comprises a positive peak (Positive Peaks, +P) and negative peak (Negative Peaks, −P), the peak positive and negative peak values appearing in the unit time reflects the characteristics of cardiomyocytes and beating cell state.

(See FIG. 17) cardiomyocytes beat patterns and associated main parameters:

Amplitude (Amplitude)

Cell-index difference between the positive and negative peaks between the peaks (shown in FIG Amp-1, Amp-2, Amp-3, -, Amp-m of the movable arch (Beating Period) :

As shown above, in a scanning period (Sweep Duration) the number of peaks is m(+P1, +P2, +P3, . . . , +Pm) ,the number of negative peaks is n(−P1,−P2,−P3, Pn). A time positive and negative peaks is defined as between the peaks of run out (Beatings Period). for each beat by the rising period (Rising Tine), fall time (Falling Time), IBD₅₀, a rising slope (Rising Slope), and the down slope (Falling Slope) and other parameters to describe the process of beating up and down.

(See FIG. 18)

3.5.3 myocardial cell model parameters and data analysis methods RTCA myocardial cell analysis software provides 12 parameters used to analyze characteristics of beating cardiac muscle cells, In this report, select the applicable parameters.

(See FIG. 19)

| Parameter | Unit | Method | Parameter Description |
|---|---|---|---|
| Beating | Beating/ | Positive Peak | AU positive peaks within |
| Rate | min | Counts | a few minutes |
| | | Negative Peak | All negative peaks |
| | | Counts | within a few minutes |
| | | Positive Peak | Calculated for each |
| | | Period Based | positive-positive |
| | | | beat frequency between |
| | | | the peaks on |
| | | | the crest of a positive. |
| | | Negative Peak | Calculated for each |
| | | Period Based | positive-positive |
| | | | beat frequency between |
| | | | the peaks on |
| | | | the crest of a negative |
| Normalized | N/A | Positive Peak | When selecting |
| Beating | | Counts | standardized beating |
| Rate | | Negative Peak | frequency, the frequency |
| | | Counts | drop-down menu appears, |
| | | Positive Peak | standardized time point |
| | | Period Based | from which to choose. |
| | | Negative Peak | Standardization beating |
| | | Period Based | frequency is the |
| | | | ratio of the beating |
| | | | frequency at |
| | | | selected time points and |
| | | | the beating frequency at |
| | | | standardized time points. |
| Amplitude | Same as | WP-Whole Peak | The jump amplitude |
| | Cell Index | | from a negative |
| | | | peak to a next positive peak. |
| | | PP-Positive Peak | The jump amplitude |
| | | | from the baseline to |
| | | | the Positive Peak |
| | | NP-Negative Peak | The jump amplitude |
| | | | from the baseline to |
| | | | the negative peak |
| Normalized | N/A | WP-Whole Peak | When selecting |
| Amplitude | | PP-Positive Peak | standardized jump |
| | | NP-Negative Peak | amplitude, the |
| | | | frequency drop-down |
| | | | menu appears, |
| | | | standardized time point |
| | | | from which to choose. |
| | | | Standardization jump |
| | | | amplitude is the |
| | | | ratio of the jump |
| | | | amplitude at selected |
| | | | time points and the |
| | | | jump amplitude at |
| | | | standardized time points. |

4. Results produced only medicine extract on cell viability at a concentration of 16 mg/ml, other concentrations had no effect on cell viability. The extract of beating cardiomyocytes impact: short term primary rat ventricular myocytes beat frequency, amplitude, and rhythm no obvious influence, longer duration of action after ventricular myocytes have certain heart toxicity. Especially in high concentrations (16mg/ml), will be in effect can cause myocardial cell parameters.

Beat frequency is reduced, the role of induced cells 6 hours after the beating stopped completely. Low concentrations of the drug (5.33 and IJ8 mg/ml) beat frequency induced myocardial cells within 2-12 hours after dosing interval and the magnitude of the increase is slightly lower, 12 child beating back to normal.

Drug concentrations below 0.59 mg/ml beating cardiac muscle cells had no significant effect.

4.1 extract of Chinese medicine in primary not neonatal cardiomyocytes Activity

(See FIG. 20)

Chinese medicine extract: (H20 ctrl, blank ct, 7.32 ug/ml, 21.95 ug/ml, 65.84 ug/ml, 0.20mg/ml, 0.59 mg/ml, 1, 78, mg/m15.33 mg/ml, 16.00 mg/ml)

Chinese medicine extract inhibited the activity of primary cardiomyocytes dynamic testing. The picture shows the cell growth and drug effects in real time dynamic map. Among them, the horizontal axis represents cell culture and drug action time; vertical axis represents the normalized cell index (NCI). Cell Index prompt cell growth state. The higher its value reflects more living cells. The arrows indicate the inter-dosing inch, as shown in Figure dosing concentration. The graph shows the concentration of the drug in the drug-dependent cell dynamic response curve, Ctrl as a negative control, showed normal cell growth dynamic curve. (Note: H20 Ctrl same control wells was added 10% (the highest concentration of drug stock solution volume) of H20) The results of primary neonatal rat cardiomyocytes visible activity of extracts from traditional Chinese medicine, a high concentration of the drug when given time (18 mg/ml), cell index declined rapidly after a period of time, reflecting the high concentration of the drug has significant cytotoxicity, can lead to cell morphological changes, poor adherence or even death. When the drug concentration is less than 5.32 mg/ml, the impact of drugs on cell activity is not obvious. But this concentration of the drug but it will cause the beating cardiomyocytes beat frequency and amplitude of a certain degree of change.

4.2 Primary beating cardiomyocytes with time after dosing curve changes

(See FIG. 21) 4.3 Chinese medicine extract primary neonatal rat cardiomyocytes toxic effects on summary

| (μg/ml) | 1600 | 5330 | 1780 | 590 | 200 | 65.84 | 21.95 | 7.32 | CON |
|---|---|---|---|---|---|---|---|---|---|
| Before | ✓ | ✓ | ✓ | ✓ | ✓ | ✓ | ✓ | ✓ | ✓ |
| treatment | | | | | | | | | |
| 10 min | ✓ | ✓ | ✓ | ✓ | ✓ | ✓ | ✓ | ✓ | ✓ |
| 30 min | ✓ | ✓ | ✓ | ✓ | ✓ | ✓ | ✓ | ✓ | ✓ |
| 1 h | ✓ | ✓ | ✓ | ✓ | ✓ | ✓ | ✓ | ✓ | ✓ |
| 2 h | X | ✓ | ✓ | ✓ | ✓ | ✓ | ✓ | ✓ | ✓ |
| 6 h | XX | X | X | ✓ | ✓ | ✓ | ✓ | ✓ | ✓ |
| 12 h | XX | X | X | ✓ | ✓ | ✓ | ✓ | ✓ | ✓ |
| 18 h | XX | X | X | ✓ | ✓ | ✓ | ✓ | ✓ | ✓ |
| 24 h | XX | X | X | ✓ | ✓ | ✓ | ✓ | ✓ | ✓ |

Note:
The effect of drugs on cell beating,
XX, no beating;
X, obvious abnormalities;
✓, normal beating.

Role 4.4 medicine extract on beating frequency (Beating rate) of

(See FIG. 22)

The picture shows the cardiomyocytes beat frequency Herbal Extracts drug concentration and time-dependent changes in dose-effect relationship. The parameters used for the standardization of beating frequency (Normalized Beating Rate, NBR), parameter calculation method for the selection of the time point of beating frequency and standardization of time (0 min) beat frequency ratio

Figure A process of change which is within the dosing 1 h cells beat frequency; Figure B drug after 10 min prior to dosing beat frequency between 24 h cells throughout the change process. Seen from the figure, within 10 minutes before dosing, cell beating amplitude stability. After a short period of drug dosing no significant effect on cell beating, 2 hr after high concentrations of drug-induced cell beating frequency is reduced until the beating stopped completely after 6 hr, 5, 33 mg/ml over a period of time due to the drug beat frequency slightly lower drug concentration less than 1.78 mg/ml no significant effect on cell beating. Have a significant impact on the description of the high concentration of the drug effect when the heart beats, low concentrations without seriously affecting the role of the heart beat.

4.5 Chinese medicine extract on the beat amplitude (Amplitude) role

(See FIG. 23)

The picture shows the magnitude of the beating cardiomyocytes drug concentration and time dependent variations. Use parameters for standardization beat amplitude (Normalized Beat amplitude ratio normalized time point Amplitude, NA), parameter calculation method for the selection of time points (0 min) beat frequency. Figure A is a process of change in which dosing 1 h cells within the beating amplitude; Figure B for drug dosing 10 min prior to dosing throughout the cell after beating amplitude variation between 24 h process.

Visible, the dosing before 10 min cells bouncing stable from the figure, after a short period of drug dosing had no significant effect on cell beating amplitude, 2 hr high concentration of 16 mg/ml after beating reduce the frequency of drug-induced cell until after 6 hr completely stopped beating, 5.33 mg/ml/drug-induced beat frequency decreased slightly higher drug concentration is less than 1.78 mg/ml no significant effect on cell beating. Have a significant impact on the description of the high concentration of the drug effect when the heart beats, low concentrations without seriously affecting the role of the heart beat.

4.6 Chinese medicine extract on neonatal rat cardiomyocytes were beating frequency and amplitude of the IC50

(See FIG. 24)

FIG. 4 time point IC50 curve fitting (beating rate/Amplitude)

| | Beating rate | | Amplitude | |
|---|---|---|---|---|
| Time after | IC50 | | IC50 | |
| dosing | (mg/ml) | Square R | (mg/ml) | Square R |
| 6 hr | 6.88 | 0.985 | 9.88 | 0.983 |
| 12 hr | 7.00 | 0.990 | 10.65 | 0.987 |
| 24 hr | 6.68 | 0.970 | 7.72 | 0.539 |

Calculate the three time points IC50 values (beating rate/Amplitude) based on cell beating frequency and amplitude. 5 Conclusions and experiment indicates possible problems and suggestions

5.1 of the drug at high concentrations (16 mg/ml) quickly affect cell viability, leading to cell death.

5.2 The Chinese influence in a concentration-dependent manner in primary neonatal rat cardiomyocytes beat frequency and amplitude. High concentration (16 mg/ml) will lead to the cells at around 6 hr after arrest, while moderate concentrations (533 mg/ml, 1 78mg/ml) can be caused when cells in about 6 hr after beating frequency slightly slower, slightly increases the magnitude Have a significant impact on the description of the high concentration of the drug effect when the heart beats, low concentrations without seriously affecting the role of the heart beat.

Specific ion channel-related myocardial 5.3 no drug-induced jitter waveform. These results are for reference only, specific mechanisms need further screening studies. Experimental record information

Stored in Essen biological (Hangzhou) Co., Ltd. customer database.

Code: 1112121715CD.

What is claimed is:

1. A pharmaceutical composition containing human cyclooxygenase and doxorubicin or a doxorubicin analogue, wherein the raw material of the composition is Rare grass.

2. The pharmaceutical composition according to claim 1 wherein the term “the raw material of the composition is Rare grass” means: using the resulting powder dried and crushed from Rare grass whole plant dehydration; or the Rare grass extract.

3. The pharmaceutical composition according to claim2, wherein the extract of the herb Treasure refers to the following manner extract one of:
Powder obtained from concentrated and dried Rare grass decoction;
Ethanol extracts obtained from Rare grass decoction by ethanol extract; or powder obtained from ethanol extractive of Rare grass decoction, and then concentrated and dried;
Organic solvent extracts obtained from Rare grass whole plant or decoction by organic solvent extraction;
or powder obtained from Organic solvent extracts of Rare grass whole plant or decoction, and then concentrated and dried;
Extracts obtained from Rare grass whole plant or decoction by supercritical carbon dioxide extraction; or powder obtained from extracts of Rare grass whole plant or decoction by supercritical carbon dioxide extraction, and then concentrated and dried;
Rare grass whole plant extract essential oil.

4. The pharmaceutical composition according to claim 1, 2, or 3, wherein the pharmaceutical composition containing human cyclooxygenase and doxorubicin further contains materials allowed by the pharmaceutical field.

5. The pharmaceutical composition according to claim 4, wherein the term “allowed by the pharmaceutical field” means: as excipients, flavoring agents or antioxidants in a tablet; as an antioxidant or stabilizer in a capsule;
act as flavoring agents, stabilizers or antioxidants in oral liquid or syrup; used as emulsifiers or when petrolatum ointment for external use; injection time as emulsifiers, solvents, or stabilizers.

6. A preparation method of the pharmaceutical compositions according to claim 1, wherein by the following steps:
(1) Rare grass whole plant cleaned;
(2) The step (1) the whole plant grass get clean Treasure dehydrated and dried to obtain pulverized powder;
Said step (2) or in one of the following steps of extraction of the herb extract Treasure:
(2)-1 Rare grass decoction, or concentrated, and dried to obtain a powder;
(2)-2 Rare grass decoction, alcohol extract obtained by ethanol extraction, or concentrated, and dried to obtain a powder;
(2)-3 Rare grass or grass decoction of the whole plant. The organic solvent extract obtained by extraction, or a concentrated and dried to obtain a powder;
(2)-4 whole plant or herb extracts Rare grass decoction obtained by supercritical car n dioxide extraction, or a re-concentrated and dried to obtain a powder;
(2)-5 Rare grass whole plant extract volatile oil;
(3). Powder and 1 or Volatile oil obtained from Step (2), the step (2)-1, in step (2) -2, in step (2) -3, in step (2)-4 or (2)-5, are mixed with pharmaceutical excipients; and are made forms required;
(4) sterilization;
(5) test;
(6) Packaging.

7. The pharmaceutical composition according to claim 1 in the preparation of a medicament for the treatment of nephritis.

8. The pharmaceutical composition according to claim 1 in the preparation of a medicament for the treatment of cholecystitis and gallbladder polyps.

9. The pharmaceutical compositions according to claim 1 in the preparation of a medicament for the treatment of tumor.

10. The pharmaceutical compositions according to claim 1 in the preparation of anti-inflammatory drugs.
